(19) United States
(12) Patent Application Publication
(10) Pub. No.: US 2007/0276245 A1
(43) Pub. Date: Nov. 29, 2007

(54) System And Method For Automated Boundary Detection Of Body Structures

(76) Inventor: Elisa E. Konofagou, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/697,573

(22) Filed: Apr. 6, 2007

Related U.S. Application Data
(63) Continuation of application No. PCT/US05/37669, filed on Oct. 17, 2005.
(60) Provisional application No. 60/619,247, filed on Oct. 15, 2004.

(51) Int. Cl. A61B 8/13

(52) U.S. Cl. 600/443

(57) ABSTRACT

A system and method for imaging the localized viscoelastic properties of tissue is disclosed. An oscillatory radiation force is applied to tissue in order to induce a localized oscillatory motion of the tissue. The phase and amplitude of the induced localized oscillatory motion of the tissue is also detected while the oscillatory radiation force is being applied. The viscous properties of the tissue are determined by a calculation of a phase shift between the applied oscillatory radiation force and the induced localized oscillatory motion of the tissue. The oscillatory force force inducing local oscillatory motion may be a single amplitude modulated ultrasound beam.

### CLAIM FOR PRIORITY TO RELATED APPLICATIONS

This application claims the benefit of U.S. Provisional Patent Application Ser. No. 60/619,247, filed on Oct. 15, 2004, which is hereby incorporated by reference in its entirety herein.

### BACKGROUND OF THE INVENTION

1. Field of the Invention

This invention relates to a system and method for automatic image processing, in particular a technique of autocorrelation of ultrasound echoes to delineate tissue regions, such as the boundary of the endocardium of a patient's heart.

2. Background of the Related Art

Echocardiography is a common diagnostic imaging modality that uses ultrasound to capture the structure and function of the heart. A comprehensive evaluation typically entails imaging the heart in several planes by placing the ultrasound transducer at various locations on the patient's chest wall. Accordingly, the echocardiogram video displays the three-dimensional heart from a sequence of different two-dimensional cross sections (also referred to herein as “views” or “scans.”). Under different views, different sets of cardiac cavities and other structures are visible. Observation of the cardiac structures in the echocardiogram videos, especially movement of the walls and chambers over time, is typically used to assist in the diagnosis of heart abnormalities.

For example, echocardiography is useful to detect irregularities in left ventricular wall motion. In order to determine this characteristic, three-dimensional (“3-D”) models of the left ventricle can be reconstructed from segmenting the two-dimensional (“2-D”) short axis scans and 2-D long axis scans from the end diastole phase to the end systole phase of the heart function. Segmentation refers to a method of separating distinct structures from each other. As is used herein, the term structure shall refer to an object or feature in an image. In imaging, it refers to the delineation of such structure in an image and, thus, its separation from other surrounding structures.

Currently, a common method to segment the left ventricle or other cardiac structures requires a clinical cardiologist to manually trace a large number borders, a very time consuming task. For example, left ventricular borders for as many as 20 2-D short axis slices and twelve 2-D long-axis slices may have to be traced in order for provide data sufficient to reconstruct a single frame of video data a 3-D left ventricle model. A dataset, such as that used in the exemplary embodiment described hereinbelow, may consist of seven frames between end diastole and end systole, thus providing the reviewing cardiologist with as many as 20×12×7 frames to manually trace, a total of 1680 frames. This task can be extremely cumbersome for even the most skilled cardiologist.

A challenge facing those attempting to automate the procedure of image recognition is the image quality of the echo videos being analyzed. Because echo videos are the result of the ultrasound interrogation of the structure of the heart, the images may be highly degraded by multiplicative noise. Moreover, the lower echogenicity of certain tissues, such as the left-ventricular cavity, further complicates the process of automating such procedures.

Therefore there is a need to develop a technique for automatic boundary detection which addresses the limitations of the prior art when faced with a large quantity of images, often having a low degree of echogenicity and a high degree of noise.

### SUMMARY OF THE INVENTION

It is an object of the current invention is to overcome the aforementioned limitations to provide an automated boundary detection technique.

Systems and methods are disclosed for the automatic delineation of the boundary of a body structure in an ultrasound video. This invention finds useful application in detection the boundaries of cardiac tissues and cavities as represented in echocardiogram images, such as the endocardium, of a patient's heart. A method includes providing an ultrasound image or signal. An autocorrelation calculation is performed on matrices representing the signals (amplitudes and phases) of the image to generate a correlation matrix of the signal, which represents the difference in echogenicity between two structures represented in the image, e.g., the ventricular cavity and the endocardium. An edge detection technique is used to obtain the boundary of the structure.

In an exemplary embodiment, an interpolation of the correlation matrix of pixel values may be performed to resize the image to the same size as the matrices of the original image. A threshold procedure may be applied to the correlation matrix to reduce the multiple levels of shading. Machine learning techniques may be applied to vary the threshold to improve the boundary detection process. Morphological operations and median filtering may be subsequently executed.

The autocorrelation procedure may be performed on successive frames. In addition, the autocorrelation procedure may be useful for determining the displacement or deformation of walls or other structures in the images being studied.

In accordance with the invention, the object of providing a automated boundary detection technique has been met. Further features of the invention, its nature and various advantages will be apparent from the accompanying drawings and the following detailed description of illustrative embodiments.

### BRIEF DESCRIPTION OF THE DRAWINGS

FIG. 1 illustrates the system in accordance with the invention.

FIG. 2 is flow chart which illustrates the stages of boundary detection procedure in accordance with the present invention.

FIG. 3 is an exemplary image obtained using the methods in accordance with the present invention.

FIG. 4 is an exemplary image obtained using the methods in accordance with the present invention.

FIGS. 5(a)-(g) are images obtained with a method according to prior art techniques.

FIGS. 6(a)-(g) are images obtained in accordance with an exemplary embodiment of the present invention.

FIGS. 7(a)-(g) are images obtained in accordance with another exemplary embodiment of the present invention.

Throughout the figures, the same reference numerals and characters, unless otherwise stated, are used to denote like features, elements, components or portions of the illustrated embodiments. It is intended that changes and modifications can be made to the described embodiments without departing from the true scope and spirit of the subject invention as defined by the appended claims.

### DETAILED DESCRIPTION OF THE EXEMPLARY EMBODIMENTS

Exemplary embodiments of the system and methods for automatic boundary recognition are described herein. Although the exemplary embodiment is directed to a technique for boundary recognition in echocardiogram videos, it is understood that the invention has application to any type of image or signal susceptible to autocorrelation techniques, as will be described in greater detail below. It is understood that the terms “images” and “signals” shall be used interchangeably to refer to any information used to represent the structures or tissues of the patient being monitored.

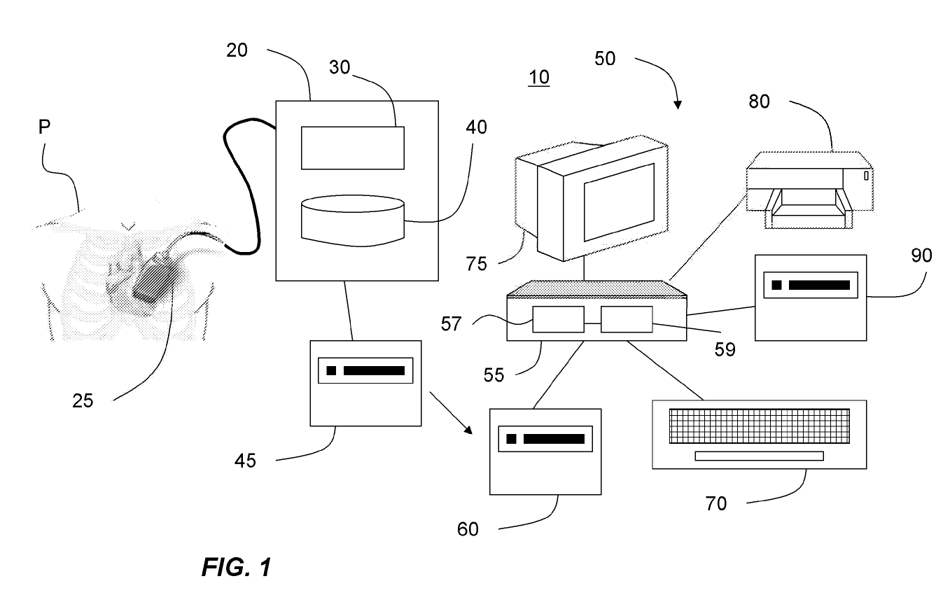

An exemplary embodiment of the system 10 is illustrated in FIG. 1, and includes signal or image acquisition equipment 20. For example, any known echocardiogram acquisition equipment, such as a 3-D Philips Sonos 7500 System having a probe 25, may be used for acquiring the images of the cardiac structure of a patient P. Image acquisition equipment may include video/signal capture equipment 30, e.g., a video capture card to digitize the analog video, and data storage equipment 40, e.g., a hard drive or other storage medium, to store the resulting video images/signals. The video images may be written onto a tape, memory card, or other medium by an appropriate recording device 45. Image processing equipment 50 is used to process the images in accordance with the invention. Image processing may be performed by a personal computer 55, such as a Dell OptiPlex GX270 Small MiniTower, or other computer, having a central processing unit or processor 57 and memory 59 storing program instructions for execution by the processor 57, an input device 60, such as tape drive, memory card slot, etc., for receiving the digital images and a keyboard 70 for receiving user inputs, and an output device, such a monitor 75, a printer 80, or a recording device 90 for writing the output onto a tape, memory card, or other medium. Image processing equipment 50 may also located on several computers, which are operating in a single location or which are connected as a remote network.

An early stage in the process is the acquisition of the datasets, e.g., echo videos, by the image acquisition equipment 20, such as the 3-D Philips Sonos 7500 System. Exemplary images include the 2-D short axis slices. Tracking the function of the heart of the patient P between end diastole to end systole is particularly useful from a diagnostic perspective because it encompasses a substantial range of contraction and expansion of the heart cavities. It is understood that any other echo views, such as the Parasternal Short Axis view or the Apical view, etc., may be used, and any portion of the heart cycle may be studied.

The automatic segmentation technique may be implemented on the image processing equipment 50 using any available computer software. In the exemplary embodiment, MATLABv6R13 was used. Cropping of the images may be performed to provide improved results. For example, the automated program may first crop the original images using the end diastole frame as a reference. This procedure assumes that the left ventricle will stay within the same coordinates from end diastole to end systole, since the left ventricle contracts during this period, and the area of the cavity is at a maximum during end diastole. The cropping may be utilized to avoid any undesired segmentation of the right ventricle. In the exemplary embodiment, the cropped images are 71×61 pixels, although other image sizes are also useful.

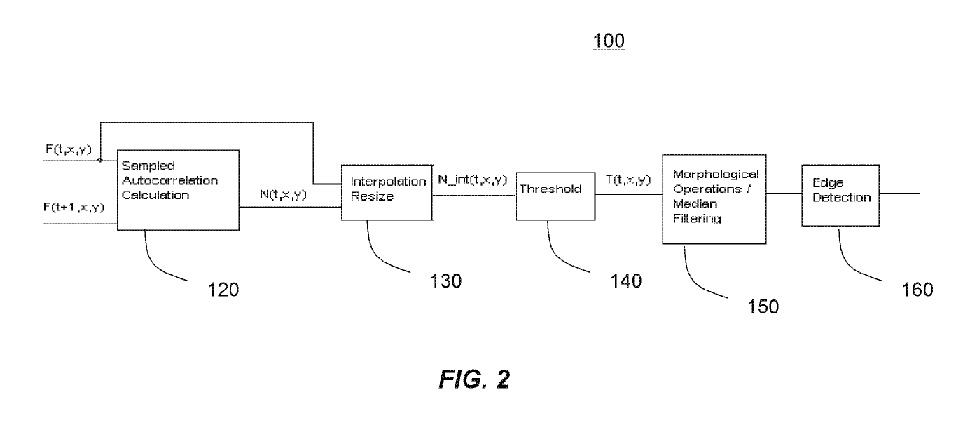

The process 100 in accordance with an exemplary embodiment is illustrated in FIG. 2. The information from two adjacent frames is used in order to find an accurate border for the structure being studied. The two frames being studied do not have to be consecutive, although such frames may preferably be reasonably close in time to ensure that the structure to be segmented has not undergone significant motion between frames. In the exemplary embodiment, it was desired to identify the endocardium of the left ventricle. Use of the autocorrelation function emphasizes the difference in echogenicity between the cavity and the myocardium of the left ventricle.

After acquisition of the images by the image acquisition equipment, another stage in the process is calculating the autocorrelation of two sampled segments from the columns of adjacent frames, e.g., frame t and the adjacent frame t+1, as indicated in equations (1) and (2):

$$\begin{matrix}{W}_{1} = {( {\sum }_{x}^{x + M} F ( t , x , y ) )}^{2} & ( 1 ) \\ {W}_{2} = {( {\sum }_{x}^{x + M} F ( t + 1 , x , y ) )}^{2} & ( 2 )\end{matrix}$$

where F(t,x,y) are the grayscale pixel values for the current frame, and F(t+1,x,y) are the grayscale pixel values for the adjacent frame. M is the size of the window in samples, x is the location along the horizontal direction of the image, and y is the location along the vertical direction of the image. “W” refers to windowed signal segment, and W₁ refers to frame t, and W₂ refers to frame t+1.

A new image may be formed by taking the inverse of a square root of these sampled autocorrelation values multiplied together (step 120), as indicated in equation (3):

$$N ( t , x , y ) = {[ {\sum }_{y = 0}^{61} \{ \sqrt{{( {\sum }_{x}^{x + M} F ( t , x , y ) )}^{2} \cdot ( {\sum }_{x}^{x + M} {F ( t + 1 , x , y )}^{2} )} \} ]}^{- 1} , \qquad ( 3 )$$

the inverse square of the regular autocorrelations. This may be used as the criterion for the threshold. In the example where the image size is 71×61 pixels, the maximum index of y is 61. (Thus, equation (3) represents an exemplary case where one dimension of pixels is 61, and this equation could be generalized for larger or smaller frames.) According to the above equations, the matrix N(t,x,y) represents a new image, which may be smaller in size than the original 71×61 pixel images. That is, N(t,x,y) will have an M number fewer rows. This is because if the window falls outside the range of the image (if x+M>71), the value of F(t,x>71,y) will not be a valid pixel value. By using a simple interpolation technique, N(t,x,y) may be resized to the same size as F(t,x,y) (step 130). Exemplary interpolation techniques are the linear or cubic interpolations. It is understood the autocorrelation procedure may be performed on a single matrix of signals values, rather than the two matrices discussed above. The autocorrelation techniques described herein may also be used to determine the motion and/or deformation of the tissue structures between frames, e.g., the wall or the cavity of the patient's heart.

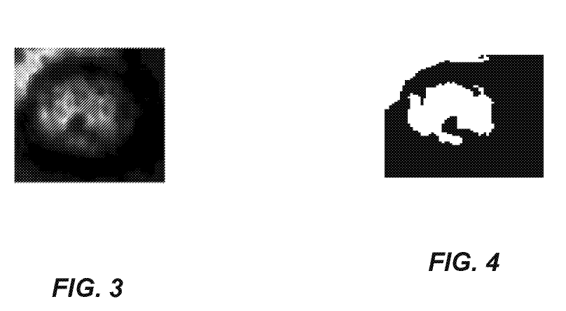

As a subsequent step, the resized matrix N(t,x,y) may then be thresholded to permit improved segmentation the left ventricle (step 140). An example of such a thresholded technique is described herein: For the cases where N(t,x,y) is less than 0.01, the autocorrelation amplitude is set to zero, while in the opposite case it is set to one. FIG. 3 illustrates an example of such an autocorrelation image 20 before thresholding. FIG. 4 illustrates the image 30 obtained after thresholding technique is applied.

Following the thresholding step, later steps of the process are basic morphological operations, e.g., a closing operation and a filling operation, to remove small artifacts resulting from the mitral valve and from papillary muscles. The ‘imclose’ and ‘imfill’ routines were applied for the closing and filling operations, respectively, using the MATLABv6R13 function ‘edge’ in order to generate a uniform surface, e.g., to merge isolated pixels, and include all pixels enclosed by the surface. These steps may also include a median filtering operation which finds the object within the image that has the largest area and removes any other objects. The above-described operations are indicated generally as step 150 in FIG. 1. With continued reference to FIG. 4, it may be seen that this operation removes pixel data inside the left-ventricular cavity 32 in FIG. 3. An edge detection is performed using the MATLABv6R13 function ‘edge’ (step 160) in order to delineate the boundary being studied, such as the endocardium.

In order to improve the boundary detection technique, the threshold value may be varied for each frame. For example, a perceptron machine learning algorithm may optionally be used. According to this procedure, the threshold is incremented by small values until the automatically detected structure is very close as determined by the best fit to that of the area calculated from a manually traced border for each frame. As with any machine learning technique, the use of more datasets of these seven frames and available datasets from previous studies, a simple machine learning algorithm can be trained to calculate optimal threshold values for each frame.

### EXAMPLE

In an exemplary embodiment, the datasets, e.g., echo videos, were acquired using a 3-D Phillips Sonos 7500 System, from a heart transplant patient at the Columbia Presbyterian Hospital. 208 2-D short axis slices were saved from end diastole to end systole. There are seven time frames between end diastole and end systole, and each 2-D slice is 160×144 pixels. In the exemplary embodiment, slice numbers 100 is used from the 208 2-D short axis slices from each time frame. This selection allowed for an easier comparison of the automatic border technique to the manually traced borders.

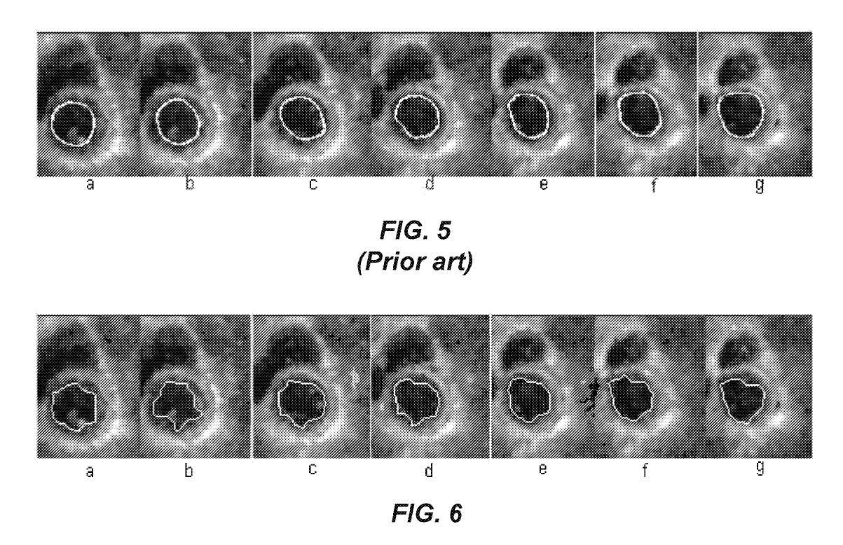

The manually traced borders were performed by a trained human observer. They were traced by using a C++ interface to a MATLABv6R13 program. The GUI interface allowed the human observer to place approximately 12 points along the border of the endocardium of the left ventricle, and the rest of the points along the border where interpolated automatically. FIGS. 5(a)-(g) illustrate the borders identified by the human observer. Each image is one time frame from the one-hundredth 2-D slice; from the first to the seventh time frame.

FIGS. 6(a)-(g) illustrate the borders traced automatically according to process 10, in accordance with the present invention. As with the manually identified images, each image is one time frame from the one-hundredth 2-D slice; from the first to the seventh time frame.

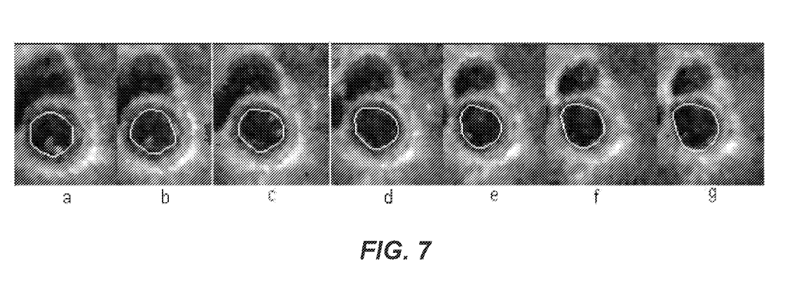

As discussed above, the threshold value may varied for each frame to aid our segmentation technique. FIGS. 7(a)-(g) illustrate the boundaries wherein the process 10, discussed above, is supplemented by a perceptron machine learning algorithm. The threshold was incremented by small values until the automatically detected ventricle area is very close to that of the area calculated from the manually traced borders for each frame.

Table 1 lists the areas calculated for each frame using the three different techniques.

TABLE 1

| | | | Relative |
|---|---|---|---|
| Border* | Frame | Area (cm²) | Error |
| M | 1 | 10.87 | — |
| A1 | 1 | 11.31 | 4.1% |
| A2 | 1 | 10.66 | 1.9% |
| M | 2 | 10.69 | — |
| A1 | 2 | 10.17 | 4.9% |
| A2 | 2 | 10.72 | 0.3% |
| M | 3 | 10.41 | — |
| A1 | 3 | 11.26 | 8.1% |
| A2 | 3 | 10.62 | 2.0% |
| M | 4 | 10.10 | — |
| A1 | 4 | 10.31 | 2.1% |
| A2 | 4 | 10.01 | 0.9% |
| M | 5 | 9.78 | — |
| A1 | 5 | 9.88 | 1.0% |
| A2 | 5 | 9.53 | 2.5% |
| M | 6 | 10.64 | — |
| A1 | 6 | 9.87 | 7.3% |
| A2 | 6 | 10.48 | 1.4% |
| M | 7 | 11.85 | — |
| A1 | 7 | 9.61 | 17.3% |
| A2 | 7 | 11.85 | 1.9% |

*A1 = automated segmentation A2 = ML (machine learning) automated segmentation M = manually detected borders
Mean relative error for A1 = 6.38%
Mean relative error for A2 = 1.57%

According to another embodiment, left-ventricular (LV) myocardial abnormalities, characterized by dyskinetic or akinetic wall motion and/or poor contractile properties, can be inferred to using myocardial elastography to assist in the automated segmentation of the left ventricle. The hypothesis is that blood and muscle scatterers have distinct motion and deformation characteristics that allow for their successful separation when motion and deformation are imaged using Myocardial Elastography (Konofagou E. E., D'hooge J. and Ophir J., IEEE-UFFC Proc Symp, 1273-1276, 2000, which is incorporated by reference in its entirety herein.)

Normal, human volunteers were scanned using a 2-MHz phased array and a Terason ultrasound scanner (Teratech, Inc., Burlington, Mass.) both in short- and long-axis views of the left ventricle. RF data were acquired over three cardiac cycles during natural contraction of the myocardium. The maximum scanning depth was 15 cm with a sampling rate of 20 MHz and an associated frame rate of approximately 20 frames/s. Corrected (or, recorrelated) two-dimensional (i.e., axial and lateral) displacement and strain estimates were imaged after using a modified, reference-independent version of a previously described technique (Konofagou E. E. and Ophir, J., Ultras Med Biol 24(8), 1183-1199, 1998, incorporated by reference in its entirety herein) that utilizes interpolation, cross-correlation and correction techniques to decouple and estimate the two main motion components. Axial and lateral, motion, deformation and correlation coefficient images were utilized and compared in order to segment the left-ventricular wall, i.e., separate the cavity region from the myocardial wall.

In both short-axis and long-axis views, during diastole, the elastograms were shown to highlight the displacement difference between the LV wall and cavity through the well-known “underline effect” that results from high gradients in the displacement. During systole, the elastograms were very noisy, mainly limited by the low frame rate used. On the other hand, during both diastole and systole, axial and lateral correlation images indicated an approximately twice higher average correlation coefficient in the LV wall compared to that inside the cavity. Contour plots of thresholded correlation coefficients, therefore, successfully delineated the borders of the LV cavity throughout all three cardiac cycles.

Even at low frame rates, two-dimensional elastographic information was shown useful in the automated differentiation between the LV wall and the LV cavity based on the fact that the cavity will deform (or, decorrelate) in a different fashion to the myocardial wall. Compared to motion and deformation, the use of correlation coefficients were shown to be the most successful in underlying the highly decorrelating cavity and assisting a simple segmentation technique to generate automated contours throughout several full cardiac cycles in two distinct views. It is expected that higher frame rates will increase the elastographic precision in systole and, thus, allow for higher resolution necessary for refined, automated tracing and better comparison to manual tracings.

It will be understood that the foregoing is only illustrative of the principles of the invention, and that various modifications can be made by those skilled in the art without departing from the scope and spirit of the invention.

What is claimed is:

1. A method for detecting the boundary of a structure in one or more ultrasound autocorrelation calculation, applying a threshold procedure to the correlation matrix. images comprising:
receiving a matrix of pixel values corresponding to said one or more ultrasound images;
performing one or more autocorrelation calculations on the matrix of signal values corresponding to the ultrasound image to generate at least one correlation matrix; and
performing an edge detection calculation to the correlation matrix to obtain the boundary of the structure in the one or more ultrasound images.

2. The method according to claim 1, further comprising, after performing an autocorrelation calculation, interpolating the correlation matrix to resize the image.

3. The method according to claim 1, further comprising, after performing an autocorrelation calculation, applying a threshold procedure to the correlation matrix.

4. The method according to claim 3, further comprising calculating the threshold value through the use of a machine learning algorithm.

5. The method according to claim 4, further comprising, after performing an autocorrelation calculation, calculating one or both of the motion and deformation of the structure using correlation techniques and then continuing with thresholding.

6. The method according to claim 1, further comprising, after performing an autocorrelation calculation, applying morphological operations to the correlation matrix.

7. The method according to claim 1, further comprising, after performing an autocorrelation calculation, applying median filtering operations to the correlation matrix.

8. The method according to claim 1, further comprising
providing matrices of pixel values corresponding to first and second ultrasound images, wherein the second image represents a condition occurring subssequent to a condition represented by said first image, and wherein the step of performing an autocorrelation calculation on the matrix of signal values comprises performing an autocorrelation calculation on the matrices of signal values corresponding to the first and second ultrasound images to generate at least one correlation matrix.

9. A system for detecting the boundary of a structure in an ultrasound image, comprising:
a processor and memory operatively couple to the processor, the memory storing program instructions for execution by the processor to receive a matrix of pixel values corresponding to one or more successive ultrasound images; to perform an autocorrelation calculation on the matrix of signal values corresponding to the ultrasound images to generate at least one correlation matrix; and to perform an edge detection calculation to the correlation matrix to obtain the boundary of the structure.

10. The system as recited in claim 9, wherein the processor is further adapted to, after performing an autocorrelation calculation, interpolate the correlation matrix to resize the image.

11. The system as recited in claim 9, wherein the processor is further adapted to, after performing an autocorrelation calculation, apply a threshold procedure to the correlation matrix.

12. The system as recited in claim 11, wherein the processor is further adapted to calculate the threshold value through the use of a machine learning algorithm.

13. The system as recited in claim 12, wherein the processor is further adapted to, after performing an autocorrelation calculation, calculate one or both of the motion and deformation of the structure using correlation techniques and then continue with thresholding.

14. The system as recited in claim 9, wherein the processor is further adapted to, after performing an autocorrelation calculation, apply morphological operations to the correlation matrix.

15. The system as recited in claim 9, wherein the processor is further adapted to, after performing an autocorrelation calculation, apply median filtering operations to the correlation matrix.

16. The system as recited in claim 9, further comprising:
image acquisition equipment for generating the matrix of pixel values corresponding to the one or more successive ultrasound images.
